(19) United States
(12) Patent Application Publication
(10) Pub. No.: US 2007/0184125 A1
(43) Pub. Date: Aug. 9, 2007

(54) Use of extracts from gentiana lutea as an antimicrobial agent

(76) Inventors: Pontus Stierna, Danderyd (SE); Michael A. Popp, Lauf-Heuchling (DE); Chaim Ismail, Neumarkt (DE)

Correspondence Address: FISH & RICHARDSON, PC, P.O. BOX 1022, MINNEAPOLIS, MN, 55440-1022, US

(21) Appl. No.: 10/570,890

(22) PCT Filed: Sep. 9, 2004
(86) PCT No.: PCT/EP04/10079

§ 371 Date: Feb. 28, 2007

(30) Foreign Application Priority Data
Sep. 9, 2003 (DE) 103415793

(51) Int. Cl. A61K 36/515

(52) U.S. Cl. 424/725; 424/773

(57) ABSTRACT

The present invention concerns a use of an extract from Gentiana lutea (yellow gentian) as a drug for the treatment of bacterial infections. It has been found that the extract exhibits an anti-microbial effect that corresponds to the effectivity of ampicillin. The novel drug may be made into any customary galenic formulations and is quite particularly suited for the treatment of infections of the upper and lower respirational tracts, such as rhinosinusitises and bronchitises.

The present invention concerns a use of an extract from Gentiana lutea (yellow gentian) for the treatment of bacterial infections in accordance with claim 1, as well as an antimicrobial agent in accordance with claim 7.

Preparations from the root of Gentiana lutea (yellow gentian) have been used for a long time in natural healing and popular medicine, respectively. Reported areas of application are the plague, gout, in neurological disorders, and as an anthelmintic. In accordance with Monografie (date of announcement: Nov. 11, 1985, date of publication in Bundesanzeiger [Federal Gazette] No. 223; 14335: Nov. 30, 1985, Correction: published in Bundesanzeiger of Mar. 13, 1990) of the Commission E of the former Federal Health Office (Bundesgesundheitsamt BGA, now: Bundesinstitut für Arzneimittel und Medizinprodukte, BfArM), the indications for the use of preparations from the root of Gentiana lutea are as follows: “digestion disorders such as loss of appetite, sensation of fullness, flatulence”.

The root of Gentiana lutea is part of the present applicant's fixed drug combination SINUPRET®. The plant drug combination has been used for decades with inflammations of the upper and lower respirational tracts. Evidence for the effectivity of the drug was furnished through several controlled clinical studies. Additional medicinally active components of the mentioned fixed drug combination are Flores Primulae cum Calycibus, Herba Rumicis, Flores Sambuci, and Herba Verbenae.

The hitherto described pharmacological effects of preparations from the root of Gentiana lutea are brought about by the bitter constituents contained in the drug. Through a stimulation of the taste receptors they reflexively result in an increased secretion of saliva and gastric juice. In the Perry & Boyd model in the rabbit, an increase in the quantity of bronchial secretion was demonstrated.

Furthermore it is generally known to treat infections in man and animal by means of synthetic or half-synthetic antibiotics. What is known, e.g., is the group of β-lactam-based antibiotics to which penicillin and ampicillin belong also. Owing to their chemical nature and enantiomer mixtures resulting from synthesis, the like antibiotics—on top of an increasing resistance of patients against the administration of chemical products—frequently have undesirable side effects.

Starting out from the prior art of the combined use of extracts from Gentiana lutea in the form of the known SINUPRET®, it accordingly is the object of the present invention to furnish a plant-based antimicrobial agent which at least approximates the effectivity of ampicillin, however has less side effects than the latter.

The above object is achieved through the features of claim 1.

The present invention concerns in particular the use of an extract from plant material of Gentiana lutea (yellow gentian) for the manufacture of a drug for the treatment of bacterial infections that are selected from the group consisting of: infections with: gram-positive bacteria, in particular clostridiae, streptococci, preferably Streptococcus pneumoniae; as well as Staphylococcus aureus, preferably methicillin-resistent Staphylococcus aureus, in a particularly preferred manner Staphylococcus aureus NCTC 11940.

It is preferred to obtain the extract from roots, leaves, or stalks of Gentiana lutea.

In a particularly preferred manner, the extract from Gentiana lutea may be used for the treatment of infections of the upper and lower respirational tracts such as rhinosinusitises, in particular sinusitis maxillaris and/or sinusitis frontalis, as well as bronchitis.

An advantageous use of the present invention is founded in using the extract from Gentiana lutea for the treatment of bacterial superinfections of primary afflictions brought about by viruses.

The present invention further concerns an antimicrobial agent which contains an extract from material of Gentiana lutea (yellow gentian).

For the purposes of the present invention, the agent, in particular drug, is manufactured by an extraction of plant material, in particular roots, by means of organic solvents, in particular polar solvents, preferably alcohols, in a particularly preferred manner ethanol, in particular mixtures of ethanol with water, preferably 50% (vol.) ethanol; or by means of super-critical CO₂, wherein the extract is obtained within a temperature range from 31° C. to 90° C. and a pressure range from 75 to 500 bar, and separation takes place within a temperature range from 10° C. to 50° C. and a pressure range from 1 bar to 74 bar.

The agent may, of course, be manufactured in the customary galenic formulations, in particular sugar-coated tablets, tablets, drops, syrup, sprays, in particular nose sprays, salves, cremes, but also as a preparation for injection.

In this connection the auxiliaries customary in the prior art are used.

In accordance with the invention, the antibiotic agent on the basis of Gentiana lutea possesses at least the antibiotic effectivity of ampicillin in the distribution of mucous membrane samples on blood agar plates in comparison with Streptococcus pneumoniae.

The extract from Gentiana lutea may, e.g., be used orally in a dose of approx. 100 mg/kg to 1000 mg/kg, in particular approx. 200 mg/kg to 500 mg/kg, preferably approx. 300 mg/kg of extract.

Hitherto anti-microbial properties have not been described in scientific literature for preparations from the root of Gentiana lutea.

The anti-microbial effect of the preparation from the root of Gentiana lutea in accordance with the invention was demonstrated in a study on the whole animal. The model used was the standard model in accordance with Naclerio [Bomer et al.: Archives of Otolaryngology—Head and Neck Surgery, 1998].

Further advantages and features of the present invention may be derived from the description of practical examples as well as by referring to the drawings, wherein:

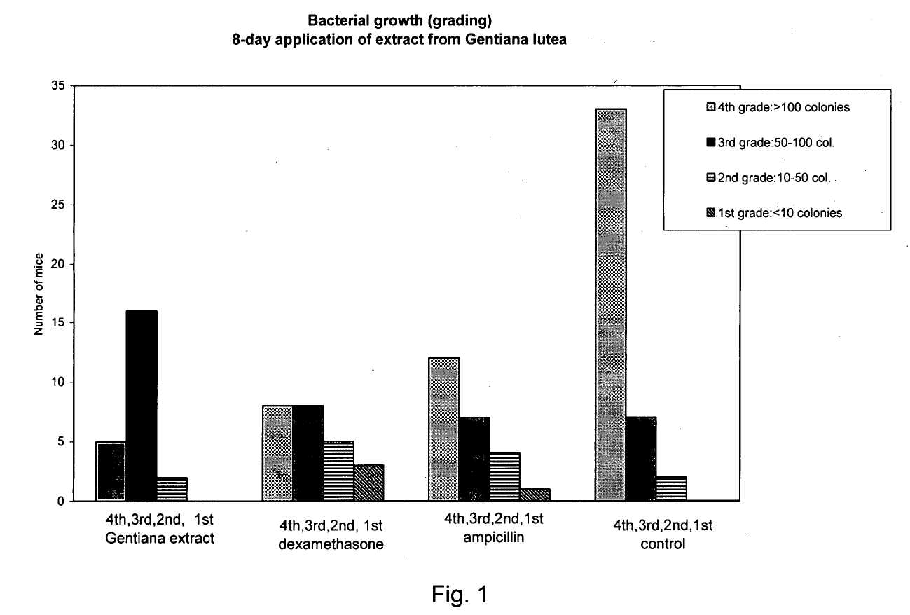

FIG. 1 is a bar chart representing the growth of bacteria following an 8-day application of an extract from Gentiana lutea in comparison with the antiphlogistic dexamethasone and the antibiotic ampicillin versus an untreated control;

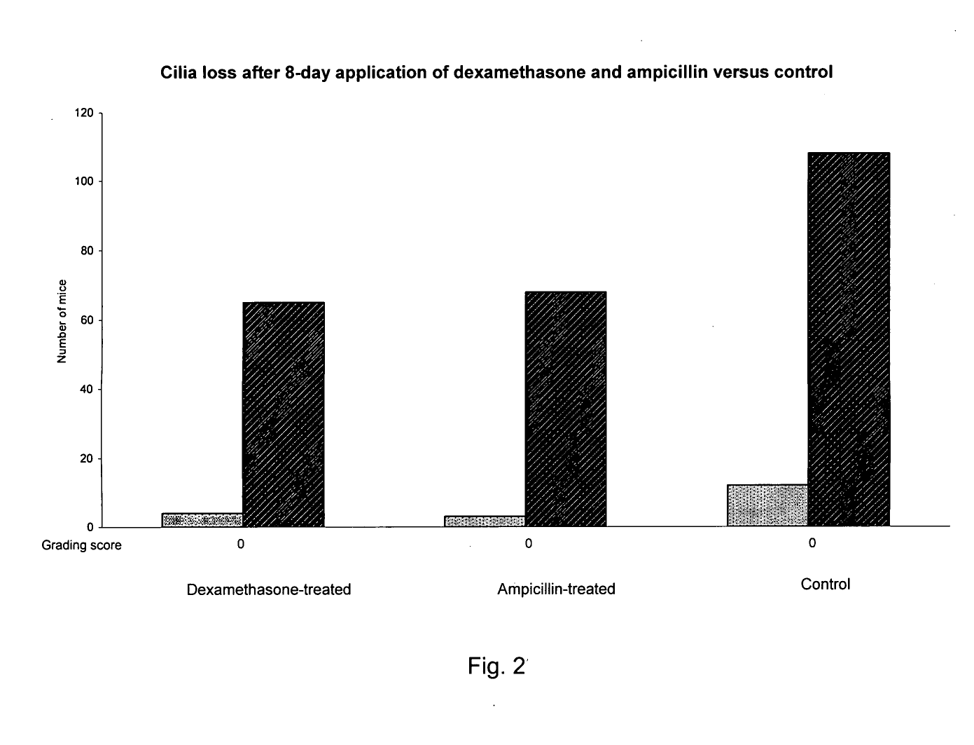

FIG. 2 is a bar chart representing the cilia loss of the mucous membranes of nose and sinuses after an 8-day application of dexamethasone and ampicillin versus an untreated control;

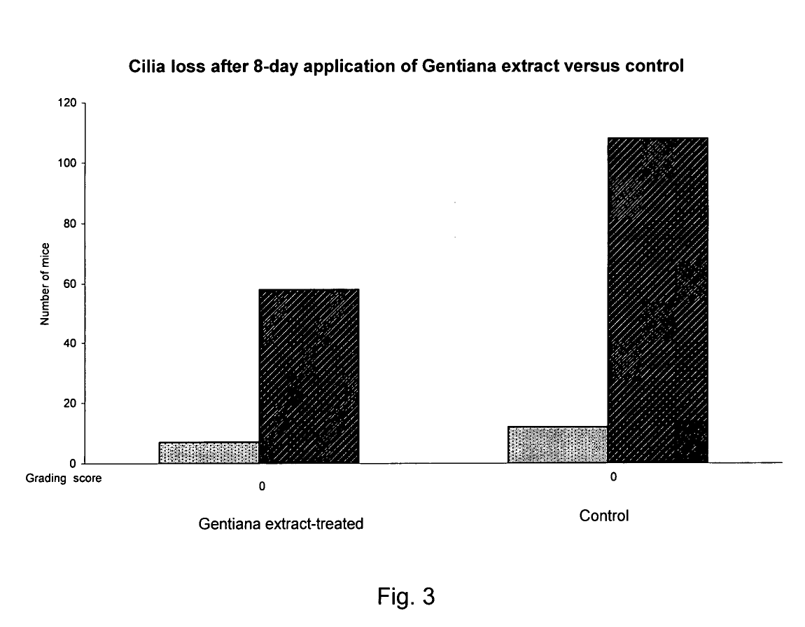

FIG. 3 is a bar chart representing the cilia loss of the mucous membranes of nose and sinuses after an 8-day application of an extract from Gentiana lutea versus an untreated control;

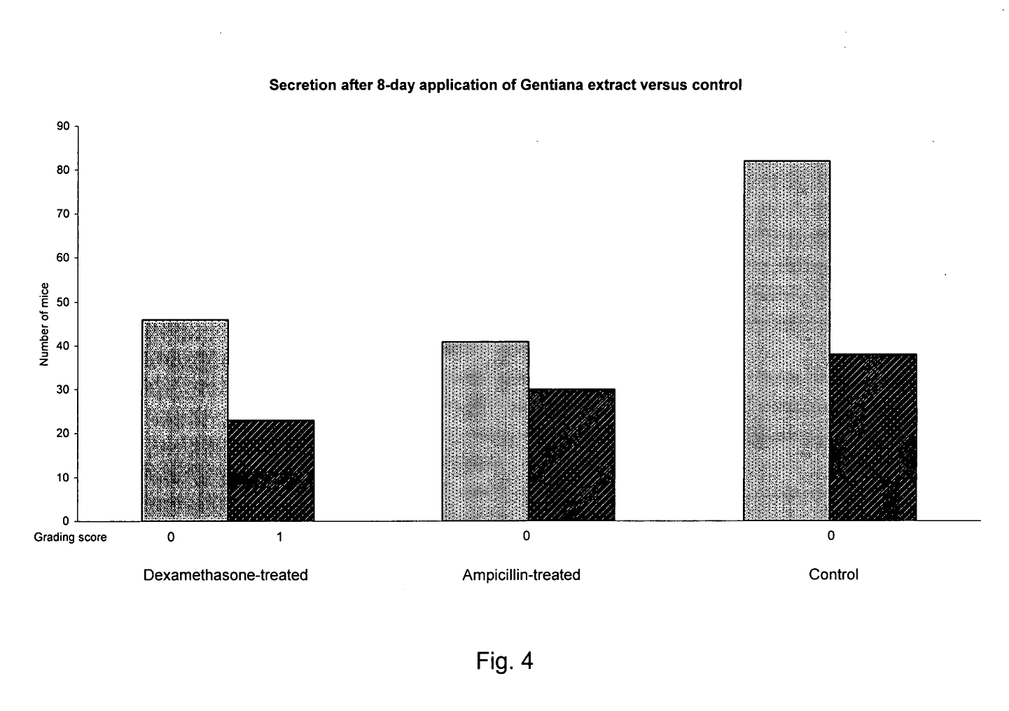

FIG. 4 is a bar chart representing the measured secretion of the mucous membranes of nose and sinuses after an 8-day application of dexamethasone and ampicillin versus an untreated control;

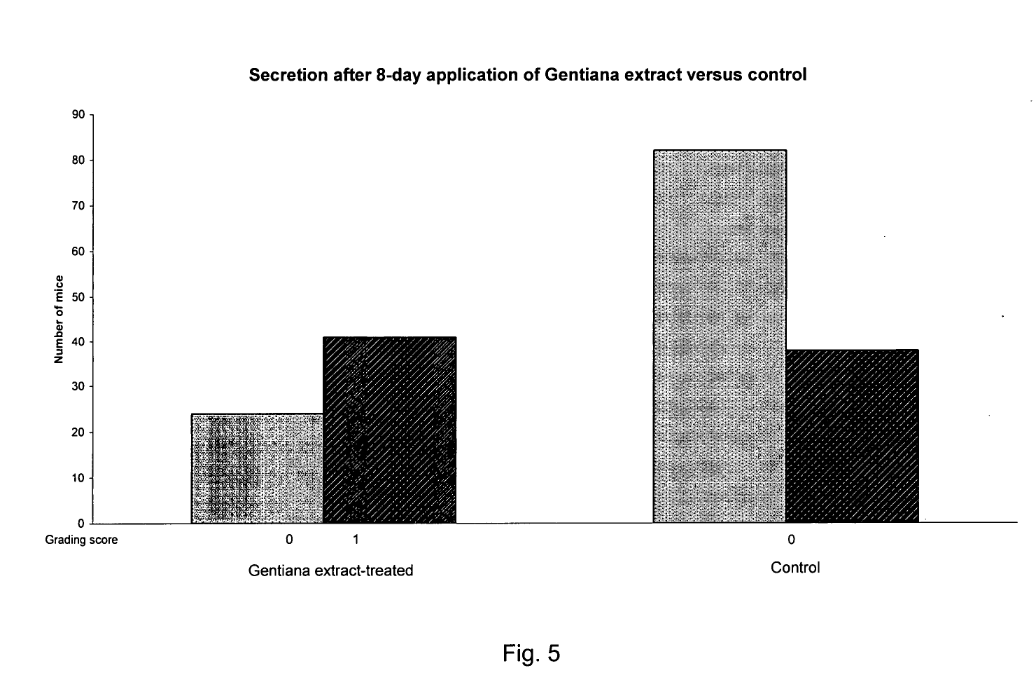

FIG. 5 is a bar chart representing the measured secretion of the mucous membranes of nose and sinuses after an 8-day application of an extract from Gentiana lutea versus an untreated control;

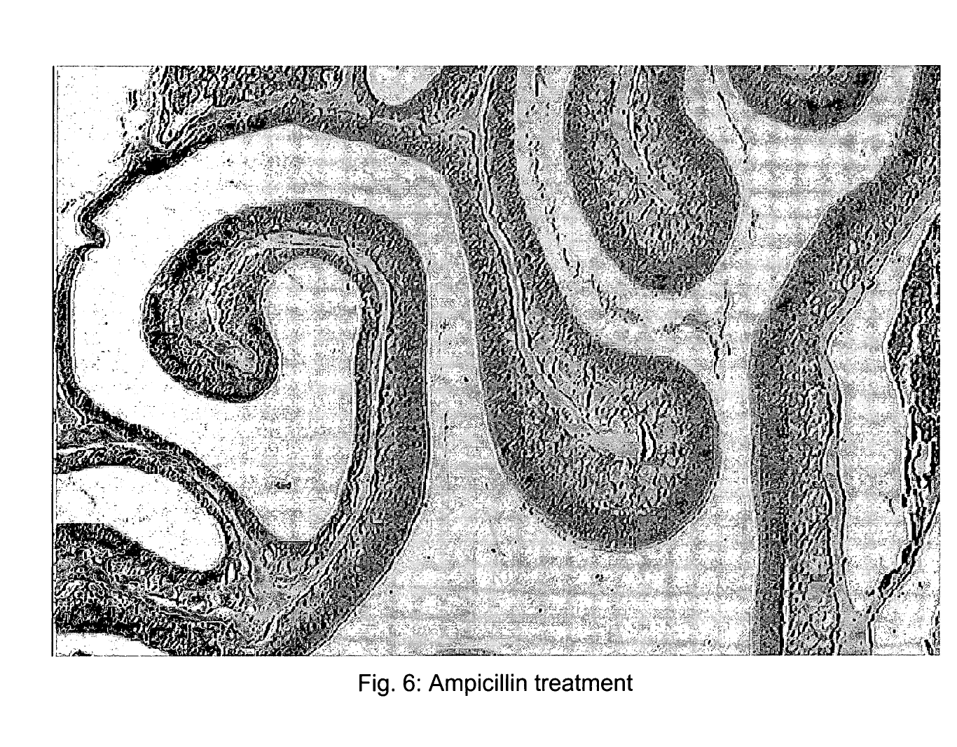

FIG. 6 is a microphotograph of a histological cut (hematoxylin-eosin staining) of nose and sinuses in black-and-white reproduction of animals that had been treated with ampicillin;

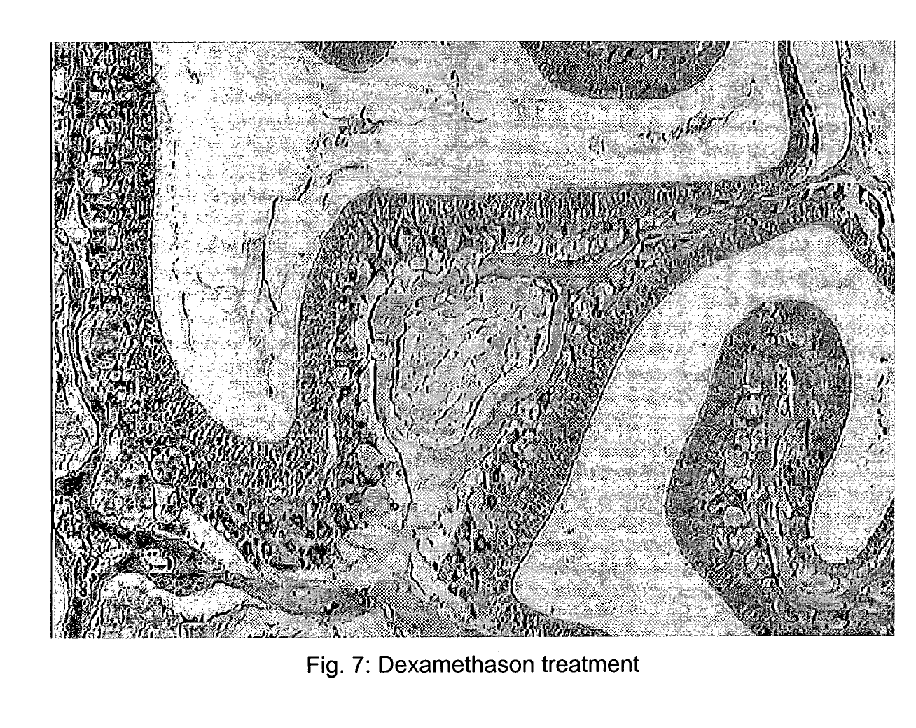

FIG. 7 is a microphotograph of a histological cut (Alcian blue-Pas staining) of nose and sinuses in black-and-white reproduction of animals that had been treated with dexamethasone;

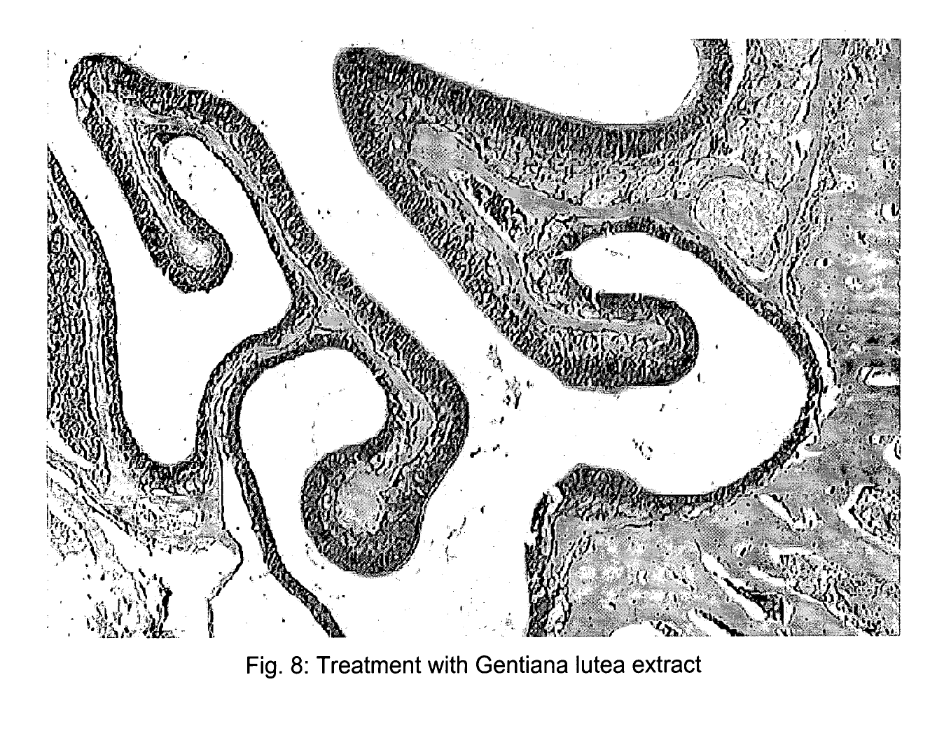

FIG. 8 is a microphotograph of a histological cut (hematoxylin-eosin staining) of nose and sinuses in black-and-white reproduction of animals that had been treated in accordance with the invention with an extract from Gentiana lutea; and

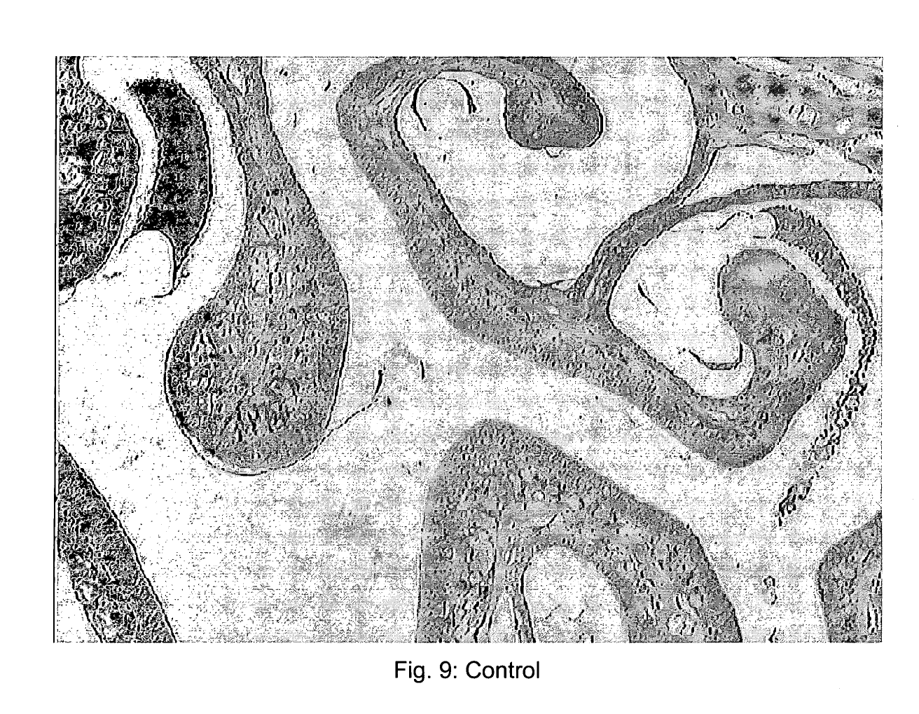

FIG. 9 is a microphotograph of a histological cut (hematoxylin-eosin staining) of nose and sinuses in black-and-white reproduction of animals that were sham-treated for control.

In the named model, BALB/c mice are infected locally with Streptococcus pneumoniae, Serotype 3, by application of the pathogen in the nose. As a result of the exposure with the bacteria, a bacterial inflammation of the nose and sinuses—a rhinosinusitis—is caused in the animals.

In the study presently described, the mice were infected in the above described manner. The mice were associated to various treatment groups in a randomized manner. The groups consisted of at least 50 experimental animals each. The mice were either sham-treated, treated with an extract from the root of Gentiana lutea, or treated with an antibiotic (ampicillin), or dexamethasone.

The extract from the root of Gentiana radix was obtained by means of extraction (ethanol concentration extracting agent 50% v/v, room temperature, duration of extraction: 7 hours) and drying (drying time 20 hours, maximum product temperature 45° C.).

Ingestion of the extract from the root of Gentiana lutea was oral, with the test substance being dissolved in a sugar solution. The administered concentration was 300 mg of extract per kg of body weight animal per day. The sham-treated animals merely ingested the sugar solution without test substance. Application of the antibiotic (300 mg per kg of body weight animal per day) was equally oral. Dexamethasone (1 μg per kg of body weight animal per day) was applied parenterally.

Treatment began respectively in parallel with induction of the bacterial rhinosinusitis. The duration of the sham treatment, of the treatment with the extract from the root of Gentiana lutea, and of the treatment with the antibiotic was 4 and 8 days, respectively. The treatment with dexamethasone was carried out only once at the day of induction of infection. One half of the animals were killed 4 days following induction of the infection, the other half were killed 8 days following induction of infection.

Examined Parameters

Bacteriological Examinations

Samples of the left and right sinus system were taken with a sterile swab and distributed directly on blood agar plates, and incubated under aerobic and anaerobic conditions at 37° C. for 48 hours.

The isolated bacteria were characterized by means of gram test and morphology. The oxidase test was performed, and the AP1 20 NE-Test (bioMerieux, France) was used for identification.

Macroscopy

Following decapitation of the mice, the macroscopic result of nose and sinuses was documented. The following classification of the results was used:
0=negative/normal result
1=swelling of the mucous membrane
2=swelling and serious secretion
3=mucoid or mucopurulent secretion
4=pussy secretion

Histology (See FIGS. 6 to 9)

Nose and sinuses were fixed in 4% paraformaldehyde, decalcified, and embedded in paraffin. The entire complex was sliced serially in 5-micron layers. Staining was carried out with two different staining methods, hematoxylin-and-eosin, Alcian blue-Pas.

The following parameters were examined semiquantitatively:
mucous membrane edema
epithelium lesion
development of mucous cells
mucous glands
infiltration of inflammation cells
   Results

Bacteriological Examinations

Surprisingly, anti-microbial effects were found in the described model for the preparation from roots of Gentiana lutea.

In comparison with the sham-treated control, after 4 days all of the named active treatment groups exhibited a tendency towards reduced growth of bacteria on the described growth media. After 8 days, all of the active treatment groups exhibited a statistically significantly reduced growth of bacteria (see FIG. 1).

Macroscopic Examinations

In comparison with the sham-treated control, after 8 days all of the named active treatment groups exhibited a statistically significantly reduced pathology of the secretion or of the nasal mucous membrane conditions, respectively (see FIGS. 4 and 5).

Histological Examinations

In comparison with the sham-treated control, after 8 days all of the named active treatment groups exhibited a statistically significantly reduced pathology concerning cilia loss (see FIGS. 2 and 3 as well as FIGS. 6 to 9).

In the ailment-specific model, an anti-microbial effect of a preparation from the root of Gentiana lutea was ascertained for the first time. These are comparable to the effects of the antibiotic ampicillin in the same whole animal model.

Infectionally caused inflammations of the upper respiratory tract (in particular rhinosinusitis) and of the lower respiratory tract (in particular bronchitis) are primarily caused by viruses. What is typical for the named ailments, however, is a superinfection with bacterial pathogens. The germ Streptococcus pneumoniae that was used in the present model belongs to the typical germs that cause a bacterial superinfection on the respiratory tracts.

Antibiotics in particular, also the ampicillin used in the present model, belong to the therapeutical options in the treatment of infections of the upper and lower respirational tracts.

1. A method of treatment of bacterial infections, comprising administering to a subject an extract from plant material of Gentiana lutea (yellow gentian), wherein the bacterial infection is a gram-positive bacteria infection.

2. The method of claim 1, wherein the extract is obtained from roots, leaves, or stalks of Gentiana lutea.

3. The method of claim 1, wherein the bacterial infection is of the upper a or lower respirational tracts.

4. The method of claim 3, wherein the bacterial infection is selected from among rhinosinusitises, maxillaris, sinusitis frontalis, and bronchitis.

5. The method of claim 3, wherein the infection is a superinfection of an ailment caused by a virus.

6. The method of claim 1, wherein the extract exhibits at least the antibiotic effectivity of ampicillin in the distribution of mucous membrane samples on blood agar plates in comparison with Streptococcus pneumoniae.

7. The method of claim 1, wherein the extract is administered in a dose of 100 mg/kg to 1000 mg/kg.

8. The method of claim 1, wherein the extract is formulated in a galenic formulation.

9. The method of claim 8, wherein the extract is formulated as a nasal spray.

10. The method of claim 1, wherein the extract is obtained by extracting plant material by means of organic solvents, in polar solvents, or by means of super-critical CO₂.

11. The method of claim 1, wherein the bacterium is a clostridiae or streptococci bacterium.

12. The method of claim 11, wherein the bacterium is a Streptococcus pneumoniae.

13. The method of claim 1, wherein the bacterium is a Staphylococcus aureus.

14. The method of claim 13, wherein the Staphylococcus aureus bacterium is methicillin-resistent Staphylococcus aureus.

15. The method of claim 14, wherein the methicillin-resistent Staphylococcus aureus bacterium is Staphylococcus aureus NCTC 11940.

16. The method of claim 7, wherein the extract is administered in a dose of 200 mg/kg to 500 mg/kg.

17. The method of claim 7, wherein the extract is administered in a dose of 300 mg/kg.

18. The method of claim 8, wherein the formulation is selected from among sugar-coated tablets, tablets, drops, syrup, sprays, salves, cremes, and a preparation for injection.

19. The method of claim 10, wherein the polar solvent is an alcohol.

20. The method of claim 19, wherein the alcohol is ethanol.

21. The method of claim 10, wherein the polar solvent is a mixture of ethanol and water.

22. The method of claim 21, wherein the mixture if 50% by volume ethanol.

23. The method of claim 10, wherein the super-critical CO₂ extraction is performed within a temperature range of 31° C. to 90° C. and a pressure range of 75 to 500 bar and separation takes place within a temperature range of 10° C. to 50° C. and a pressure range of 1 bar to 74 bar.

24. A method of preparing an extract from plant material of Gentiana lutea (yellow gentian), comprising:
extracting the plant material in an extracting agent comprising an organic solvent, a polar solvent or super-critical CO₂.

25. The method of claim 24, wherein the extracting agent is super-critical CO₂ and the extraction is performed within a temperature range of 31° C. to 90° C. and a pressure range of 75 to 500 bar and separation takes place within a temperature range of 10° C. to 50° C. and a pressure range of 1 bar to 74 bar.

26. The method of claim 24, wherein the extracting agent is an alcohol.

27. The method of claim 24, wherein the extracting agent is a mixture of alcohol and water.

28. The method of claim 27, wherein the extracting agent is 50% ethanol (vol/vol).

29. The method of claim 24, wherein the plant material is the roots, leaves or stalks of Gentiana lutea.

30. The method of claim 29, wherein the plant material is the roots of Gentiana lutea.

31. The method of claim 28, wherein:
the plant material is the roots of Gentiana lutea; and
the duration of extraction is 7 hours.

32. The method of claim 31, further comprising drying the extracting agent at a temperature such that the maximum extract temperature is 45° C.

33. A pharmaceutical composition, comprising
an extract from Gentiana lutea formulated for delivery as a nasal spray.
